(19) United States
(12) Patent Application Publication
(10) Pub. No.: US 2025/0195865 A1
(43) Pub. Date: Jun. 19, 2025

(54) Material Characteristics Ideal for Providing Either Partial or Total Mechanical Support to the Failing or Arrested Heart and Method for Developing Ideal Characteristics for Underlying Cardiac Disorders

(71) Applicant: Lifebridge Technologies LLC, Dayton, OH (US)

(72) Inventor: Mark P. Anstadt, Kettering, OH (US)

(21) Appl. No.: 19/067,788

(22) Filed: Feb. 28, 2025

Related U.S. Application Data
(63) Continuation-in-part of application No. 18/150,746, filed on Jan. 5, 2023, now Pat. No. 12,263,332.
Application No. 18/150,746 is a continuation-in-part of application No. 17/931,853, filed on Sep. 13, 2022.

(51) Int. Cl. A61M 60/268

(52) U.S. Cl. CPC A61M 60/268

(57) ABSTRACT

A system and method for determining the proper dynamic strain profile of an elastomeric construct. The strain characteristics of a deficient heart are determined and compared to the normal strain characteristics of a healthy heart. A construct having elastomeric elements is provided that can expand along multiple axes. In an unloaded condition remote from the deficient heart, the elastomeric elements are pressurized to determine the pressure differential being experienced. Furthermore, optimal strain characteristics are calculated along a first axis and a second axis as a function of the pressure differential. The first optimal strain characteristic and the second optimal strain characteristic are used to estimate the dynamic strain characteristics that will be applied to the heart. Using an automated drive, the dynamic strain characteristics are compared to the optimal strain characteristics required by the heart to determine if the construct is proper.

### RELATED APPLICATIONS

This application is a continuation-in-part of U.S. patent application Ser. No. 18/150,746, filed Jan. 5, 2023, which is a continuation-in-part of U.S. patent application Ser. No. 17/931,853, filed Sep. 13, 2022.

### BACKGROUND OF THE INVENTION

### 1. Field of the Invention

In general, the present invention relates to the methodology of creating and/or selecting an elastomeric construct with the proper dynamic strain profile for a given application within the body. More particularly, the present invention relates to the methods of determining the needed strain profile of an in vivo elastomeric construct, such as an elastomeric construct used in or outside the heart and matching the strain profile with materials and designs that will provide the needed characteristics.

### 2. Prior Art Description

In biomedical engineering, strain is a measure of how a material changes in dimensions when acted upon by a force. Since a length is a distance between two points in a plane, the measure of strain is relative to the plane in which it occurs. When forces are applied to a three-dimensional object, the forces create strains in multiple planes. The resulting strains created by such forces act in all planes and are referred to as being three-dimensional strains. The three-dimensional strains that occur in a material as a result of an applied force are said to be the strain dynamics of that material.

When acting upon tissue within the body, the strain dynamics supplied by an implanted construct can be either passive or active. Passive strain dynamics occur when the implanted construct is simply imposed or attached to tissue in the body. The implanted construct imposes some resistance to the natural strains of the contacted tissue. On the other hand, active strain dynamics can occur when an implanted construct actively transfers forces to tissue. In such a case, external forces are imposed onto the implanted construct which in turn are transferred to the tissue, therein creating a dynamic strain interaction with the tissue. These forces can be applied to the implanted construct in repetitive cyclic fashion to provide a therapeutic effect on the tissue.

When an implanted construct is used to transfer a force to biologic tissue, the details of the construct's three-dimensional strain dynamics have important implications. Specifically, the strain dynamics of the construct influence the manner in which such forces are transferred to the tissue. Unfavorable strain dynamics can cause adverse consequences such as improper distribution of forces and tissue trauma. Whereas favorable strain dynamics transfer forces as intended without tissue trauma.

A body is filled with various organs, muscles, and other tissue that expand and/or contract. As the organ, muscle, and/or tissue expands and/or contracts, the organ, muscle, and/or tissue undergoes various strains that act in a variety of directions. The conformational, three-dimensional milieu of the various strains can be considered the dynamic strain profile for the organ, muscle, and/or tissue. The strain profile of a certain organ, muscle or tissue can be directly measured by applying strain gauges to such features. Alternatively, the strain profile of a certain organ, muscle or tissue can be measured using data from X-ray scans, MRI scans, ultrasonic scans, or other strain scans that image tissue within the body. Furthermore, for organs like the heart, the dynamic strain profile can be estimated using physical models, virtual models, and/or statistical models.

Given the anatomy, gender, and age of a person, an optimal dynamic strain profile can be readily determined for most any organ, muscle, tissue, or portion thereof. If an organ, muscle, or tissue that is diseased, injured, or damaged, the strain profile of the afflicted organ, muscle, or tissue will differ from the optimal dynamic strain profiles. The issue for the healthcare provider is how to fix the inflicted organ, muscle, or tissue so that the actual dynamic strain profile can better resemble the optimal dynamic strain profile.

In many situations, the dynamic strain profile of an organ, muscle, or tissue can be improved by applying an elastomeric construct to the organ, muscle, or tissue. The elastomeric construct has its own dynamic strain profile. The goal is to select or create an elastomeric construct so that when forces are applied to the construct, the construct will react with a dynamic strain profile that compensates for the deficiencies in the actual dynamic strain profile. Furthermore, the elastomeric construct should embody the ideal surface-to-tissue interaction and/or attachment for the functionality of transferring strain to the organ, muscle, or tissue being affected.

The situation becomes more challenging when the elastomeric construct contains compartments that actively apply forces. For example, an elastomeric heart cuff may be placed outside the ventricles of the heart to assist the heart in contracting and expanding. The physical presence of the heart cuff around the heart has an effect on the heart. Furthermore, in such an application, forces from an external drive are applied to the heart cuff. The structure of the heart cuff and the external drive forces applied to the heart cuff should combine to embody dynamic strain characteristics that affect the heart in a manner that is beneficial to the pumping action of the heart. However, consideration must also be given to maintaining an appropriate surface-to-tissue interaction in order to facilitate the transfer of desired strain characteristics in a non-traumatic fashion and to promote healthy recovery of tissue function.

The actual dynamic strain profile of the functioning heart when pumping blood can be measured. This dynamic strain profile represents a relatively load independent measure of the heart's pumping function. The dynamic strain profile can be used to determine the degree of pumping function and assess the degree of function and/or dysfunction in a variety of disease states. Dynamic strain profiles for optimally functioning hearts can therefore be used as targets when determining the strain therapy needed by diseased hearts.

It is important that heart cuffs and similar devices are constructed of elastomeric materials that embody strain dynamics beneficial to the heart. The heart cuff's mechanical forces should be distributed in a manner that promotes pump function while not harming tissue or negating underlying pump function. A heart cuff's strain profile, when acting on the heart, is dictated by the material characteristics, the resistive forces of the underlying heart, and the therapeutic forces exerted by the drive system. These dynamic forces vary over the entire heart pumping cycle. The forces reverse directionality from augmenting the heart either filling (diastole cycle) versus emptying (systole cycle). The dynamic strain profiles of the heart cuff act in many different dimensions to properly transfer forces to the heart's surface. While these strain profiles may be viewed and/or represented within a single plane or direction, the three-dimensional characteristics of the cuff's strain profile are critical for the proper delivery of mechanical forces to the surface of the heart. Although the elastomeric heart cuff may embody the proper dynamic strain profile in one directional plane it needs to be designed such that it has the proper dynamic strain profiles in all dimensions to optimize the mechanical pump function of the underlying heart. The forces must also impact the heart in a non-traumatic fashion as to avoid injury to the underlying tissue.

An ideal heart cuff must provide the proper dynamic strain characteristics through both the systolic and diastolic cycles of the heart, prevent tissue trauma, and facilitate improved pump function of the dysfunctional and/or non-beating heart. Therefore, the elastomeric construct ideally possesses strain characteristics that meet these requirements. Also, an ideal heart cuff should have a strain profile that can optimize strain dynamics of the functioning myocardium, thereby promoting recovery. This becomes more important when intrinsic heart muscle function is capable of meaningful contraction. When considering both of these requirements, the elastomeric construct used in the heart cuff should provide mechanical pump function to the non-beating, arrested (e.g., ventricular fibrillation) heart while also permitting recovery of underlying myocardial contractile function. Such dynamic strain characteristics of the heart cuff might be considered ideal when mirroring the epicardial surface strain patterns during optimal pump function.

The dynamic strain characteristics of a heart cuff are opposite to the dynamic strain characteristics of the heart muscle during the transfer of mechanical systolic and diastolic forces. The exception is at the end of diastole when the heart reaches maximal diastolic dimensions the elastomer may expand to further expand the ventricular myocardium of the heart. In other words, during systolic force application, the elastomeric heart cuff generally exhibits positive strain as it stretches over the heart. Conversely, during ventricular systole, the heart generally exhibits negative strain as it expels blood and/or the muscle contracts. During diastolic force application, the elastomeric heart cuff exhibits negative strain as it returns to its unstrained position. However, some degree of positive strain may be used in late diastole as the elastomeric heart cuff can further expand the heart. This late diastolic expansion facilitates maximal filling of the heart's end-diastolic capacity, thereby further improving pump function. This is noteworthy when considering that heart cuffs are known to potentially impair diastolic filling of the heart. The heart muscle exhibits positive strain throughout diastole as the ventricular myocardium expands and/or lengthens to accommodate blood entering the heart's chambers.

A need therefore exists for an elastomeric heart cuff and pump that best enables forces to be transferred to the heart to produce ideal ventricular pump function. The elastomer utilized should enable the transfer of mechanical forces that enable the heart muscle to pump in a three-dimensional fashion that results in the optimal pump function. One condition is that of ventricular fibrillation and/or asystole, in which the heart muscle has no intrinsic force generation. Another condition is that in which the heart has some contractile function but requires assistance to adequately pump blood. In both of these conditions, the heart has different underlying characteristic strain patterns. In fibrillation, the heart muscle is being pushed and pulled with strain patterns dictated principally by the force of the heart cuff material and by the underlying resistances of the heart muscle and blood being pumped. In the condition in which the heart has some meaningful pump function, the underlying muscle may pump best when allowed to follow the natural patterns. These contractile patterns of the functioning myocardial cells can be described as a synchronous three-dimensional strain pattern that is distinct from the strain patterns of the asystole or fibrillating heart being mechanically pumped. Notably, the unique properties defined in this disclosure provide material characteristics that allow the underlying heart to optimally pump in either state and provide both systolic (compressive) and diastolic (expansion or dilation) forces that promote the most ideal underlying ventricular strain patterns for pump function while avoiding tissue trauma.

Once an elastomeric construct is discovered that meets the intended criteria for use inside the body, it can be tested in an unloaded condition to best define its physical strain profile. In an unloaded condition outside the body, the elastomeric construct is not in contact with the heart or other tissue that apply the tissue's forces to the construct. Accordingly, an elastomeric construct can be tested under a simpler, controlled or unloaded conditions. Accordingly, one can validate the redesign elements of the elastomeric construct when unloaded. In a loaded condition, the elastomeric construct is in vivo or is attached to a functional model ex vivo which requires load characteristics to be standardized. As such, it is far more difficult to test, and/or validate a desired strain profile under loaded conditions.

There are a variety of elastomeric materials that have been approved for use inside the body. Each of these elastomeric materials have various characteristics that can be selectively adjusted by varying formulations and/or manufacturing variables. The characteristics that can be altered include hardness, tensile strength, tensile modulus, elongation, resilience, compression set, tear resistance, abrasion resistance, and specific gravity. Each of these characteristics affect how the elastomeric material reacts to applied forces. Currently, it is difficult to predict or otherwise model what elastomeric materials will best match the dynamic strain characteristics that must be embodied by a heart cuff. Testing a design in a loaded condition will eventually lead to the design that functions as desired. However, the in vivo setting is not practical for defining the material strain characteristics since the tissue interaction can confound such analyses. Likewise, testing a design in a loaded condition on a lab model uses estimations that may be less than ideal. The best solution is for there to be a way to accurately test an elastomeric construct in an unloaded condition so that the design of the construct can be customized to the particular needs of the condition that the construct has demonstrated effectiveness within the body.

The present invention is an improved method of determining the proper characteristics of an elastomeric construct while in an unloaded condition and selecting the best materials and design to suit the need. This need is met by the present invention as described and claimed below.

### SUMMARY OF THE INVENTION

The current invention describes the discovery of an ideal cardiac construct that embodies dynamic strain characteristic for applying mechanical forces to the ventricles of the heart. The discovery includes a means for assessing the material's strain dynamics in a test platform that allows the material characteristics to be reproduced and validated prior to its application for biologic use. The discovery provides a means to further develop related materials for applications in different cardiac disease states.

The present invention is designed for use on hearts of generally normal anatomic proportion and muscle mass with overall normal morphologic proportions but can be translated into hearts of diseased states. The present invention system and method determines the dynamic strain profile of a construct, such as a heart cuff. The dynamic strain profile is used in customizing a heart pump system to the specific needs of a deficient heart. To address the needs of a deficient heart, the strain characteristics of the deficient heart are first determined. The deficient strain characteristics are compared to the normal strain characteristics of a healthy heart to determine any strain discrepancies between the deficient strain characteristics and the normal strain characteristics.

The construct has elastomeric elements that expand along multiple axes when pressurized. The multiple axes include a first axis and a second axis. In an unloaded condition remote from the failing heart, the elastomeric elements are pressurized to determine the pressure differential being experienced. Furthermore, a first optimal strain characteristic is calculated along a first axis and a second optimal strain characteristic along a second axis as a function of the pressure differential.

The first optimal strain characteristic and the second optimal strain characteristic are used to estimate the dynamic strain characteristics that should be embodied by the construct. The dynamic strain characteristics that are estimated for the construct are compared to the optimal strain characteristics required by the failing heart to determine if the construct is proper for use on the failing heart. If the construct is ineffective, features of the construct are altered until the construct can provide the assist required by the heart to achieve a more optimal heart pump function.

### BRIEF DESCRIPTION OF THE DRAWINGS

For a better understanding of the present invention, reference is made to the following description of exemplary embodiments thereof, considered in conjunction with the accompanying drawings, in which:

FIG. 1 is a schematic of an exemplary embodiment of a heart pump system showing a heart, a heart cuff, and an external automated drive;

FIG. 2 shows the heart cuff of FIG. 1 in cross-section and in an unloaded condition with indicators of maximum and minimum deflections due to applied pressure differentials;

FIG. 3 shows the heart cuff of FIG. 1 from above, unloaded and in a state of maximum positive pressure inflation and producing inflated compartments;

FIG. 4 is a heart cuff illustrated by a transection of the long axis and the short axis on a compartment membrane this serves to illustrate how partial cuffs can be used to support a more limited select area;

FIG. 5 is a graph that illustrates the change in volume in a compartment as a function of pressure change from the external automated drive;

FIG. 6 is a graph that illustrates an example strain profile of an unloaded cuff across one heart pumping cycle;

FIG. 7 is a graph of material peak strain verses surface area with results for an unloaded condition, an in vivo loaded condition, and a mock loaded condition;

FIG. 8 is a graph that illustrates the relationship between active membrane surface area in relation to the maximal diameter of the heart at the end of the diastolic cycle;

FIG. 9 is a graph that illustrates the changes in pressure experienced by the heart cuff in operation;

FIG. 10 is a schematic of an exemplary embodiment of a heart pump system showing a heart in conjunction with a partial heart cuff;

FIG. 11 is a graph showing an inverse relationship between the forces applied to the heart and recovering heart function; and

FIG. 12 is a schematic of an exemplary embodiment of a heart pump system showing a heart in conjunction with a heart cuff and a heart function feedback system.

### DETAILED DESCRIPTION OF THE DRAWINGS

Although the present invention system and methodology can be embodied in many ways, only two examples are illustrated and described. The exemplary embodiments being shown are for the purposes of explanation and description. The exemplary embodiments are selected in order to set forth some of the best modes contemplated for the invention. The illustrated embodiments, however, are merely exemplary and should not be considered as limiting when interpreting the scope of the appended claims.

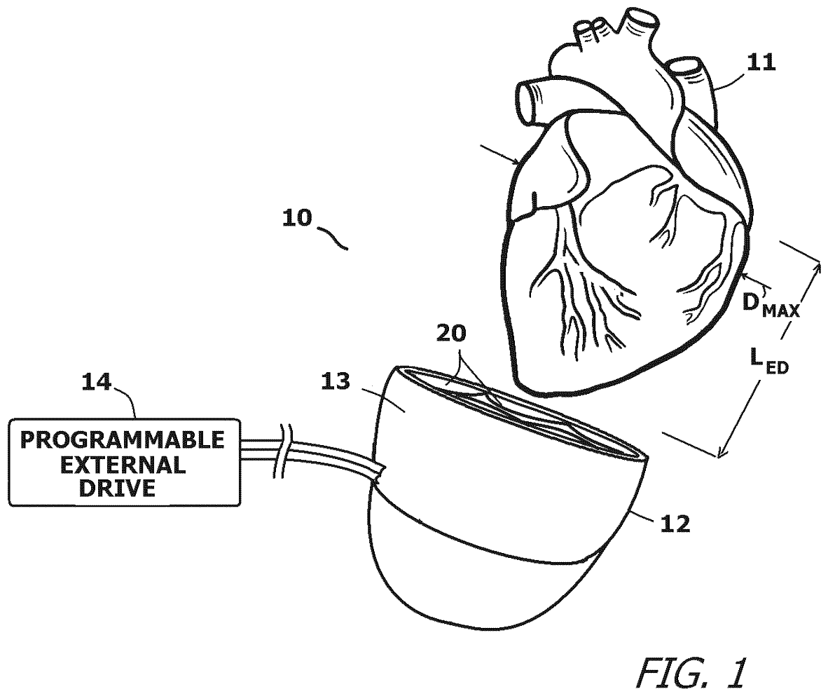

Referring to FIG. 1, a heart pump system 10 is shown that helps a heart 11 pump blood. The heart pump system 10 has an external automated drive 14 that selectively applies positive and negative pneumatic pressure to a heart cuff 12. The heart 11 has measurable dimensions that are unique for a particular individual or condition. One of the measurable dimensions is the maximal diameter of the heart in its short axis (Dₘₐₓ). The maximal diameter corresponds to the diameter of the myocardium as measured at the end of the diastolic cycle. The maximal diameter occurs on the heart below the AV groove ant a distance approximately ⅓ the distance from the AV groove to the ventricle apex. Likewise, the heart 11 has a measurable length L_{(ED)} that corresponds to the length of the ventricles at the end of the diastolic cycle. These dimensions of the heart 11 can be readily obtained from various medical scanning equipment, such as x-rays, ultrasounds, MRIs, and the like. Furthermore, it is understood that the heart 11 has a heartbeat, wherein the heart 11 contracts with a regular rhythm. Although the rhythm can be irregular, it has a general average rate of contraction over increments of time. The rhythm of the heart 11 can also be quantified using heart monitoring equipment, such as a blood pressure monitor or an ECG unit.

The heart cuff 12 fits outside the ventricles of the heart 11. In the shown embodiment, the heart cuff 12 covers the ventricles of the heart 11. However, other cuff and cup designs can be used that only contact specific areas of the ventricles. The selection of a full cuff or a partial cuff depends upon the needs of the patient. A full cuff, as shown, can apply forces to both the left and right ventricles. A partial cuff or similar device, such as a contact bladder, may only apply forces to one ventricle or part of one ventricle.

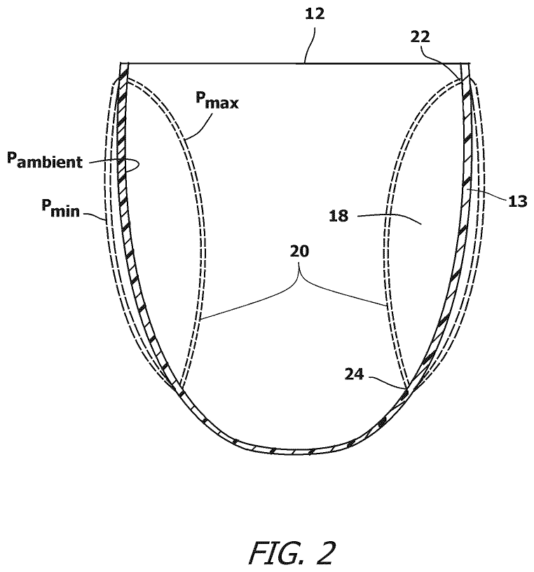

Referring to FIG. 2 in conjunction with FIG. 1, it will be understood that the exemplary heart cuff 12 being illustrated is made, at least in part, from elastomeric material. The heart cuff 12 is connected to the external automated drive 14 that can cause the heart cuff 12 to selectively contract and expand. The heart cuff 12 has an elastomeric shell 13. Within the shell 13 are a plurality of elastomeric compartment membranes 20. The expansion and contraction actions of the heart cuff 12 are typically created by applying positive and negative pneumatic pressure to various elastomeric compartment membranes 20 embodied or attached within the shell 13.

When the heart cuff 12 is applied to the heart 11, it is considered to be in a loaded condition. That is, the heart cuff 12 is being contacted by the heart 11 and is affected by the various forces applied by the heart 11. In an unloaded condition, the heart cuff 12 is not in contact with the heart 11 or any model of the heart 11. Rather, the heart cuff 12 is free to expand and contract as determined only by the design of the heart cuff 12 and the pressures applied to the heart cuff 12 by the external automated drive 14.

In FIG. 2 the heart cuff 12 is shown in an unloaded condition. As will be explained, the heart cuff 12 is designed, tested, and customized in this unloaded condition. Both the shell 13 of the heart cuff 12 and the elastomeric compartment membranes 20 within the shell 13 have the ability to expand and contract. Due to the design of the heart cuff 12, when the elastomeric compartment membranes 20 are pressurized, they tend to expand inwardly and fold in into relatively defined compartments 18. The compartments 18 are defined between the shell 13 and the compartment membranes 20. Constructs of a heart cuff 12 can be designed such that they comprise only one or multiple compartments 18 depending on how much surface area of the heart 11 in which the cuff 12 intents to act upon.

A basal attachment seam 22 is located at the top of each compartment membrane 20 where the compartment membrane 20 is anchored to the shell 13 of the heart cuff 12. Likewise, an apical attachment seam 24 is located at the bottom of each compartment membrane 20 where the compartment membrane 20 is anchored to the shell 13 of the heart cuff 12. Between the basal attachment seam 22 and the apical attachment seam 24, the compartment membrane 20 is free to expand and contract in an elastic manner. For a heart cuff sized for an average person, the surface area of compartment membranes that is free to expand and/or contract over the majority of the entire heart's outer surface is approximately 220 square centimeters, +/−55 square centimeters.

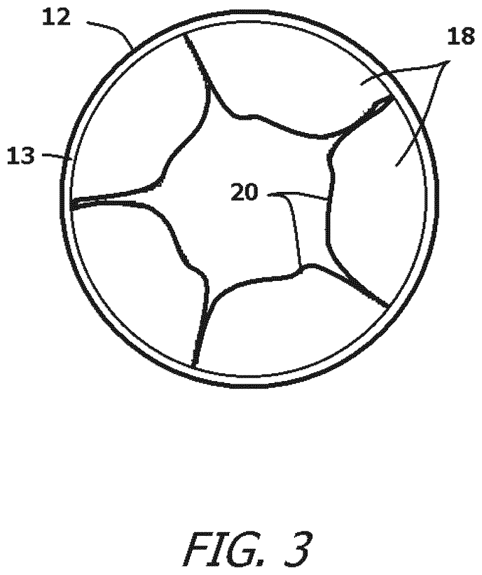

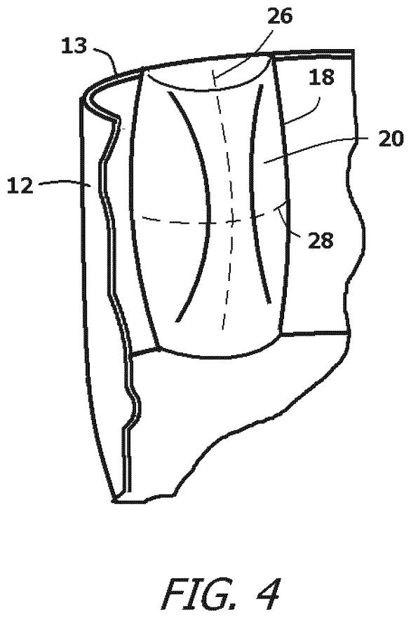

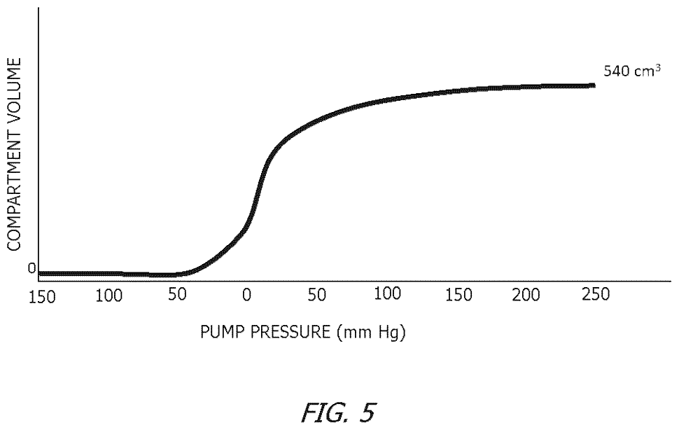

The compartment membrane 20 is made of elastomeric material that expands and contracts as different pressures are applied. Accordingly, the volume of the underlying compartment 18 dynamically changes with changes in pneumatic pressure. Referring to FIG. 4 and FIG. 5 in conjunction with FIG. 2 and FIG. 3, it can be understood that each compartment 18 has a given volume at ambient pressure. The volume increases significantly when the compartment 18 experiences a positive pressure and decreases slightly in volume when a negative pressure is applied. The changes in volume over time correspond to the change in shape of the compartment 18. The change in shape over time of the compartment 18 can be expressed as the dynamic strain ε(t) of the compartment.

The compartment 18 is a three-dimensional construct that experiences strain in multiple directional planes that include a long axis plane 26 and a short axis plane 28. The long axis plane 26 would be the long axis of the heart's ventricular surface for which the cuff is intended to act upon. The short axis plane 28 would be the short axis plane of the heart's ventricular surface for with the heart cuff 12 is intended to act upon. Strains also occur in a radial axis plane. The radial axis plane would be the radial axis of the heart's ventricles for which the cuff is intended to act upon. Measurements of strains along the long axis plane 26 and the short axis plane 28 can be used as surrogates in estimating the overall three-dimensional strains. The long axis plane 26 extends generally vertically through the center of the compartment membrane 20. The short axis plane 28 extends generally horizontally through the center of the compartment membrane 20. Accordingly, the long axis plane 26 and the short axis plane 28 are perpendicular, or near perpendicular planes that are within five degrees of perpendicular. When in an unloaded condition, the compartment membrane 20 in the long axis plane 26 and in the short axis plane 28 have initial lengths at ambient pressure. As the compartment membrane 20 expands and contracts, there is a change in length (ΔL). This change in length translates to dynamic strain ε(t), as is later explained.

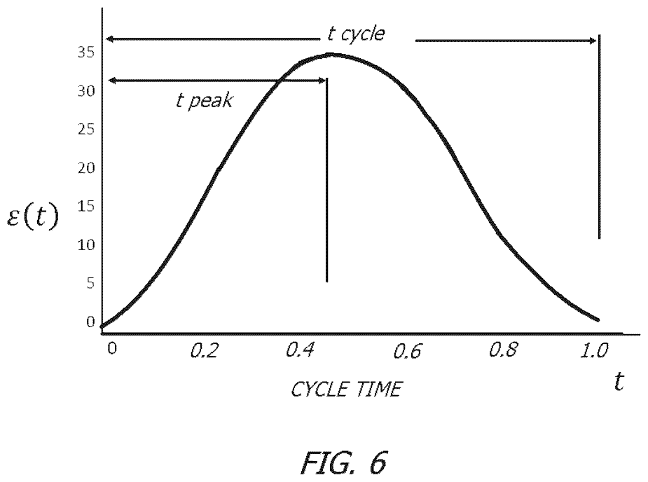

It will be understood that the compartment membrane 20 will be exposed to positive pressures and negative pressures in a manner that corresponds to the pumping rhythm of a heart. Referring to FIG. 6 in conjunction with FIG. 1 and FIG. 2, a typical ventricular strain profile for an average human heart is shown, wherein the heart is average size and morphology for the adult human population. Such a ventricular strain profile can be generated for any heart of a given diameter. As can be seen, the dynamic strain ε(t) has a compression phase and a retraction phase 40 over time (t). As can also be seen, there is a peak strain ε₍ₚₑₐₖ₎, a time to peak strain t₍ₚₑₐₖ₎, and an overall cycle period t_{(cycle)}.

The elastomeric compartment membranes 20 of the heart cuff 12 apply direct forces to the heart 11 since the compartment membranes 20 physically contact with the heart 11. The shell 13 of the heart cuff 12 applies forces indirectly to the heart 11 since the shell 13 does not directly contact the heart 11 and forces are transferred through the elastomeric compartment membranes 20. The combined forces provided by the shell 13 and compartment membranes 20 and automated drive should optimally produce an ideal strain in the heart 11 that does not damage the heart and assists the heart in achieving an optimal heart pumping functionality. An ideal strain ε(t) characteristic can be estimated using the following equation:

$$Strain = ε ( t ) = \frac{Δ L}{{L}_{0}} = \{ \begin{matrix}\frac{{ε}_{peak}}{2} ( sin ( \frac{π}{{t}_{peak}} \times t - \frac{3}{2} π ) - 1 ) \\ \frac{{ϵ}_{peak}}{2} ( sin ( \frac{π}{{t}_{cycle} - {t}_{peak}} \times ( t - {t}_{peak} ) - \frac{π}{2} ) - 1 )\end{matrix} \qquad Equation 2$$

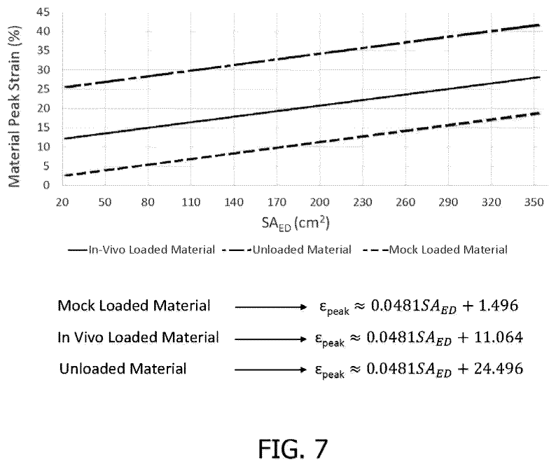

This ideal strain characteristic can be converted into a practical value for validation or testing in a construct that is being used either ex vivo in an unloaded condition, in vivo in a loaded condition, and/or in a mock loaded condition. Referring to FIG. 7, it will be understood that the ideal strain ε(t) can be converted into peak strain ε₍ₚₑₐₖ₎ for an unloaded condition using Equation 3 below:

$${ε}_{peak} \approx 0.0481 ( {SA}_{ED} ) + 24.496 \qquad Equation 3$$

This value can be adjusted by +/−10% depending upon variabilities pertaining to intended surface area of the heart or the number of cuff compartments being tested.

The ideal strain ε(t) can be converted into peak strain ε₍ₚₑₐₖ₎ for an in vivo loaded condition using Equation 4 below:

$${ε}_{peak} \approx 0.0481 ( {SA}_{ED} ) + 11.064 \qquad Equation 4$$

This value can be adjusted by +/−10% depending upon testing variables.

Lastly, the ideal strain ε(t) can be converted into peak strain ε₍ₚₑₐₖ₎ for a mock loaded condition, i.e., use on a model, using Equation 5 below:

$${ε}_{peak} \approx 0.0481 ( {SA}_{ED} ) + 1.496 \qquad Equation 5$$

This value can be adjusted by +/−10% depending upon physical variability of the mock system utilized for testing.

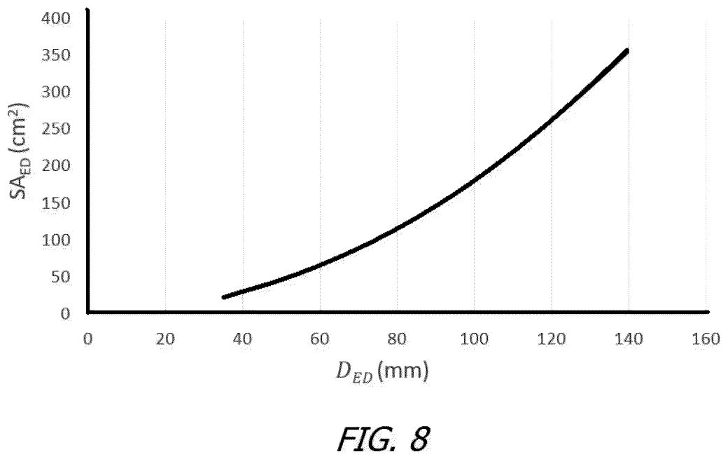

For Equation 3, Equation 4 and Equation 5, the variable SA_{ED} is the surface area of the compartment membrane 20 at the end of the diastolic cycle. To get a result from any of the equations, a value for SA_{ED} must be obtained. As can be seen from FIG. 8, the surface area of the compartment membrane 20 at the end of the diastolic cycle SA_{ED} is a function of the maximal diameter Dₘₐₓ of the patient's heart at the end of the diastolic cycle. Accordingly, by measuring the maximal diameter Dₘₐₓ of the patient's heart at the proper cycle time, the needed surface area SA_{ED} of the compartment membrane 20 becomes known.

Referring back to FIG. 1, FIG. 3, and FIG. 4, it will be understood that good estimates of the dynamic strain profile of the heart cuff 12 can be accurately estimated by knowing the strains acting in the long axis plane 26 and in the short axis plane 28 of the compartment membrane 20. The strain in the long axis plane 26 is the longest vertical deformation of the compartment membrane 20 between the basal attachment seam 22 and the apical attachment seam 24. The short axis plane 28 is the longest horizontal path over the compartment membrane 20. The degree of deformation in the long axis plane 26 and in the short axis plane 28 both depend upon the pressures supplied by the external automated drive 14. However, the pressure supplied by the external automated drive 14 may be changed to a cycle that matches the rhythmic pumping cycle of the heart 11 depending on the functioning of the heart.

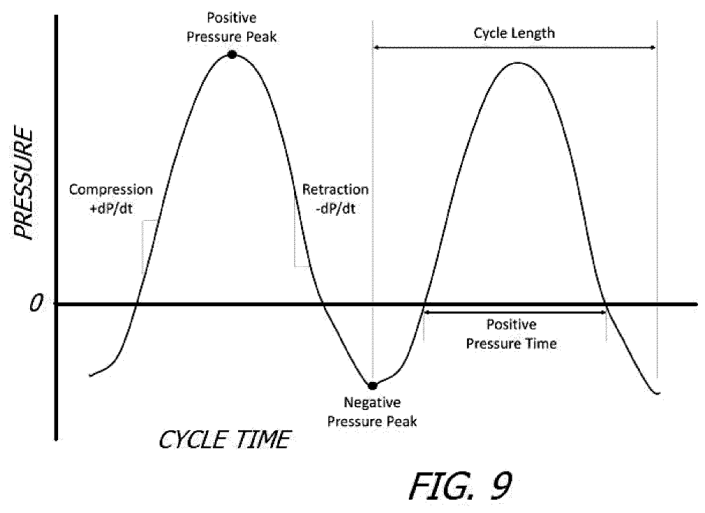

Referring to FIG. 9, it can be seen that the pressures supplied to the compartment membrane 20 by the external automated drive have a maximum pressure Pₘₐₓ, a minimum pressure Pₘᵢₙ, a cycle time T_{cycle}, and a pressure range P_{range}, which is the difference between the maximum pressure Pₘₐₓ and the minimum pressure Pₘᵢₙ during the cycle time T_{cycle}. Given these parameters the pressure P(t) on the compartment membrane at any time (t) in the cycle time can be calculated using Equation 6 below:

$$P ( t ) = \{ \begin{matrix}\frac{{P}_{range}}{2} sin ( \frac{π}{{t}_{peak}} \times t - \frac{π}{2} ) - 2 ( {P}_{min} ) & 0 \leq t < {t}_{peak} \\ \begin{matrix}( {P}_{range} ) sin ( \frac{π}{{t}_{cycle} - {t}_{peak}} \times \\ ( t - {t}_{peak} ) - \frac{3}{2} π ) - {P}_{min}\end{matrix} & {t}_{peak} \leq t < {t}_{cycle}\end{matrix} \qquad Equation 6$$

Once the pressure (Pₜ) is calculated, the stress experienced by an unloaded compartment membrane 20 in both the long axis plane 26 and the short axis plane 28 can be calculated.

The first step in calculating strain in the long axis plane 26 and in the short axis plane 28 is to use the pressure (P) to determine the maximum strain in the long axis plane 26 and in the short axis plane 28. The maximum strain ε_{(peak-LA)} in the long axis plane 26 is determined by Equation 7 below:

$${ε}_{peak - LA} = 11.676 ln ( P ) - 5.2073 \qquad Equation 7$$

The maximum strain ε_{(peak-SA)} in the short axis plane 28 is determined by Equation 8 below:

$${ε}_{peak - SA} = 6.5167 ln ( P ) - 5.1506 \qquad Equation 8$$

Again, the values generated by Equation 7 and Equation 8 can vary by up to 5% depending on testing platform and area of material and/or compartments used in the cuff. Once the peak strains are calculated in the long axis plane 26 and in the short axis plane 28, the dynamic strains can be calculated. The dynamic strain ε₍ₓ₎ for the long axis plane 26 is determined by Equation 9 below:

$$ε ( x ) = \frac{{ε}_{peak - LA}}{40.8} ( 4 \times {10}^{- 6} {x}^{4} - 0.0008 {x}^{3} + 0.0324 {x}^{2} + 0.6868 x - 0.4757 ) \qquad Equation 9$$

The dynamic strain ε₍ₓ₎ acting in the short axis plane 28 is determined by Equation 10 below:

$$ε ( {x}_{SA} ) = \frac{{ε}_{peak - SA}}{20.1} ( - 0.00005 {x}^{3} + 0.0004 {x}^{2} + 0.517 x + 0.0052 ) \qquad Equation 10$$

In view of the equations provided above, the strain profile for the compartment membrane 20 in the heart cuff 12 are calculated in both the long axis plane 26 and in the short axis plane 28.

Using the above, the strain profile for the compartment membrane 20 is calculated in both the long axis plane 26 and in the short axis plane 28 assuming an unloaded condition. This is highly useful in creating an elastomeric construct that will function very close to what is needed. However, once the elastomeric construct is loaded, that is applied to the heart, actual strain features can be determined by measurements during trial and error.

The strain dynamics embodied by the elastomeric material of the heart cuff 12 can be selectively altered by changing the thickness and/or material properties of the elastomeric material used in the heart cuff 12. The value for the dynamic strain applied by the external automated drive 14 can be controlled to some degree by active programming. However, certain variables, such as the rate that pressure can be increased and decreased is limited by the hardware being used. Accordingly, using the present invention methodology, a designer now has the ability to program the external automated drive 14 to settings that are the closest to ideal. The final changes in strain that are unachievable by programming can be achieved by altering the physical characteristics of the compartment membrane 20. By altering the elastomeric material used in the construction of the compartment membrane 20 and optimizing the programming of the external automated drive 14, the combined factors can create a dynamic strain profile that reinforces the heart 11 and enable the heart to pump in a more optimal manner.

Referring to all figures, it will be understood that the first step in determining what heart cuff 12 to use and what programming to use on the external automated drive 14 is to determine the dynamic strain profile for an average heart with generally normal morphology. The actual dynamic strain profile for the heart is measured using known techniques. The patient's heart may be in full or partial failure. The difference between the dynamic strain profile of the actual heart 11 and the dynamic strain profile of an average heart with generally normal morphology are determined. It is this difference that has to be compensated for using the heart cuff 12.

A heart cuff 12 is selected having a dynamic strain profile that was estimated and/or measured in an unloaded condition and extrapolated for the loaded condition. The heart cuff 12 is then loaded. That is, placed into contact with the heart 11. If the strain dynamics embodied by the heart cuff 12 does not provide the pumping assist needed, then the heart cuff 12 is altered or is replaced by another heart cuff that is better suited to the circumstances. The strain characteristics of the heart cuff 12 can be altered by altering the elastomeric material used in the shell 13 and compartment membranes 20 of the heart cuff 12. The elastomeric material can be made thicker and/or thinner in various places. Other variables, such as the hardness, tensile strength, tensile modulus, elongation, resilience, compression set, and specific gravity can also be altered to change the dynamic strain profile of the heart cuff.

Once the dynamic strain profile of the heart cuff 12 is altered, the altered heart cuff can again be tested. This cycle of alteration and checking can be repeated until the dynamic strain profile embodied by the heart cuff 12 matches what is needed by the actual heart in order to enable the heart to pump more in a more effective manner.

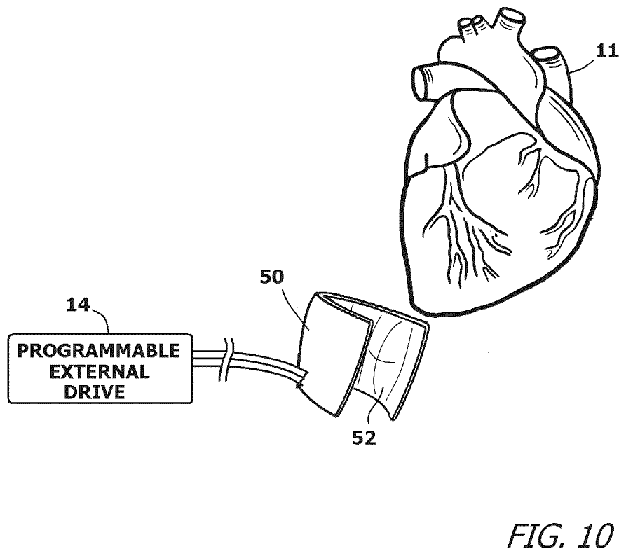

In the illustrated embodiment, the heart cuff 12 encircles the heart. It will be understood that there are many elastomeric constructs that are selectively inflated with pneumatic pressure, that do not encircle the heart. Rather, such devices typically press against only one area of the heart or are positioned internal of the heart. Referring to FIG. 10, a partial cuff 50 is shown. The partial cuff 50 can be positioned to apply forces to only the left ventricle, the right ventricle, or portions thereof. Such partial cuffs can be used when scar tissue or other abnormalities prevent the application of a full cuff. Such partial cuffs are also easier to use during non-invasive procedures. Regardless, the partial cuff 50 contains one or more elastomeric constructs 52 that apply forces to the heart 11. The methodology previously described for use of a full cuff is still valid for use on a partial cuff. Accordingly, the system and method described for full cuffs apply equally to partial cuffs or even smaller inflatable constructs. All such constructs are intended to be covered within the scope of the claims.

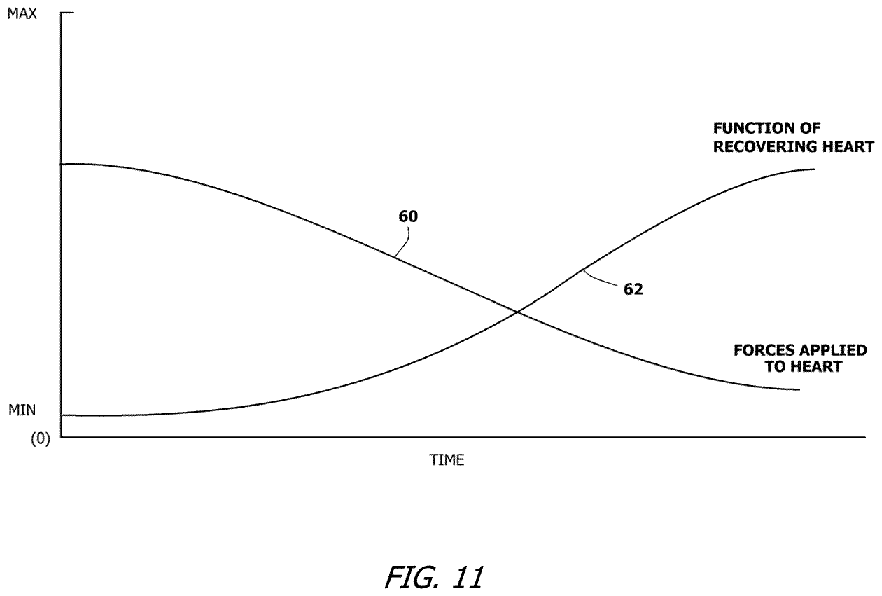

Heart cuffs are typically applied to a heart that is pumping highly inefficiently or on a heart that has stopped pumping altogether. As the heart cuff applies forces to the heart, the heart often begins to recover and pump better. Accordingly, the pumping efficiency of the heart increases during the period of time that the heart cuff acts upon the heart. If the heart cuff is powered by an external drive, which is typically a pneumatic pump. The heart cuff embodies the aforementioned dynamic strain characteristics that combine with the pressure profile provided by the external drive to apply physical forces to the deficient heart. Referring to FIG. 11, it will be understood that initially, the pressure profile supplied to the heart cuff is calculated to supplement the pumping of a stopped heart or a deficient heart at its weakest state. However, as the heart itself starts to recover, the forces 60 that are applied to the heart via the pressure profile acting upon the heart cuff does not remain constant. Rather, to prevent damage to the heart and to optimize the ability of the heart to recover, the applied forces 60 decreases in reverse proportion to the increasing heat function 62 of the recovering heart.

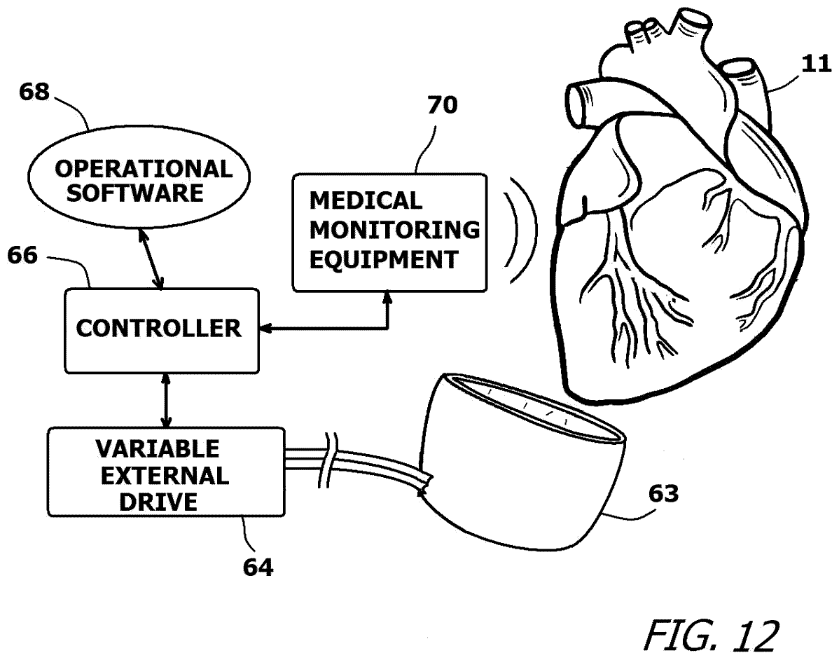

Referring to FIG. 12 in conjunction with FIG. 11, a heart cuff 63 is shown that is powered by a variable external drive 64. The heart cuff 63 has the dynamic strain characteristics previously described in earlier embodiments. The variable external drive 64 is controlled by a controller 66 that runs operational software 68.

The heart 11 is initially stopped or is pumping erratically and/or inefficiently. The heart cuff 63 is applied to the deficient heart 11. The initial deficient heart 11 is evaluated and the heart cuff 63 is operated in a manner that assists the deficient heart 11 in pumping in a more efficient manner. As previously mentioned, the initial deficient heart 11 is evaluated using medical scanning equipment, such as x-rays, ultrasounds, or the like. The deficient heart 11 can also be monitored using medical monitoring equipment, such as an electrocardiogram or pulse monitor. Medical scanning and/or monitoring is maintained while the heart cuff 63 is in place. The medical scanning and/or monitoring equipment 70 is used to determine natural heart function outside the forces being provided by the heart cuff 63. The output of the medical scanning and/or monitoring equipment 70 is directed to the controller 66. The operation software 68 utilizes the data from the medical scanning and/or monitoring equipment 70 to control the variable external drive 64 and the forces applied to the deficient heart 11 by the heart cuff 63. In this manner, the operational software 68 can be sure that the forces generated by the heart cuff 63 are synchronized with the natural rhythm being established by the recovering heart 11. Furthermore, the operation software 68 can ensure that the natural contractions of the heart 11 when combined with the forces provided by the heart cuff 63 do not cause the heart 11 to over contract or over expand during the systolic and diastolic periods of the pumping cycle.

It will be understood that the embodiments of the present invention that are illustrated and described are merely exemplary and that a person skilled in the art can make many variations to those embodiments. All such embodiments are intended to be included within the scope of the present invention as defined by the claims.

What is claimed is:

1.-19. (canceled)

20. A method of customizing a heart pump system to the needs of a deficient heart, said method comprising the steps of:
referencing average strain characteristics for an average human heart of average size and morphology;
measuring strain characteristics in vivo of said deficient heart to determine deficient strain characteristics;
comparing said deficient strain characteristics to said average strain characteristics;
providing an external drive;
providing a construct having elastomeric features that are selectively altered by said external drive;
placing said construct in contact with said deficient heart in vivo, wherein said construct, as altered by said external drive, embodies dynamic strain characteristics that produce forces that act upon said deficient heart, wherein said dynamic strain characteristics combine with said deficient strain characteristics to provide the deficient heart with overall strain characteristics closer to said average strain characteristics.

21. The method according to claim 20, wherein said external drive provides a pressure differential to said construct that cause deformations in said elastomeric features.

22. The method according to claim 21, further including measuring inherent strain characteristics of said construct when said construct is unloaded and not in contact with said heart.

23. The method according to claim 22, wherein measuring said inherent strain characteristics includes measuring deformations in said elastomeric features caused by said pressure differential.

24. The method according to claim 23, wherein measuring said inherent strain characteristics includes measuring a first axis strain from a first long axis and a second axis strain from a second short axis, where said first long axis is angled relative to said short axis.

25. A method of customizing a heart pump system to the needs of a deficient heart, said method comprising the steps of:
measuring strain characteristics of said deficient heart to obtain deficient strain characteristics;
comparing said deficient strain characteristics to statistically average strain characteristics of a healthy heart;
providing a construct having an elastomeric shell and inflatable elastomeric membranes wherein said shell and said inflatable elastomeric membranes embody dynamic strain characteristics that produce forces that act upon said deficient heart, wherein said dynamic strain characteristics combine with said deficient strain characteristics to provide the deficient heart with overall strain characteristics closer to said statistically average strain characteristics.

26. The method according to claim 25, further including measuring inherent strain characteristics of said construct when said construct is unloaded and not in contact with said heart.

27. The method according to claim 26, wherein said inflatable elastomeric membranes each have a long axis and a short axis, wherein said method further includes measuring said inherent strain characteristics by measuring a first axis strain from a first long axis and a second axis strain from a second short axis, where said first long axis is angled relative to said short axis.

28. The method according to claim 25, wherein said construct has a first axis and a second axis and wherein said method further includes calculating a first optimal strain characteristic along said first axis as a function of said pressure differential;
in said unloaded condition, calculating a second optimal strain characteristic along said second axis as a function of said pressure differential;
utilizing said first optimal strain characteristic and said second optimal strain characteristic to estimate said dynamic strain characteristics.

29. The method according to claim 28, wherein calculating a first optimal strain characteristic along said first axis includes multiplying a log of said pressure differential times a first constant and subtracting a second constant.

30. The method according to claim 28, wherein calculating a second optimal strain characteristic along said second axis includes multiplying a log of said pressure differential times a first constant and subtracting a second constant.

31. The method according to claim 28, wherein said first axis is angled relative to said second axis.

32. The method according to claim 31, wherein said first optimal strain characteristic is a function of a first peak strain measured along said first axis.

33. The method according to claim 32, wherein said second optimal strain characteristic is a function of a second peak strain measured along said second axis.
